(19) United States
(12) Patent Application Publication
(10) Pub. No.: US 2005/0283060 A1
(43) Pub. Date: Dec. 22, 2005

(54) Hematocrit automated determination device

(76) Inventors: Isakov S. Pekker, Tomsk (RU); Oleg S. Umanskiy, Tomsk (RU); Konstantin S. Brazovskiy, Tomsk (RU); Artem L. Testov, Tomsk (RU)

Correspondence Address: Angenehm Law Firm, Ltd., P.O. Box 48755, Coon Rapids, MN, 55448-0755, US

(21) Appl. No.: 10/872,586

(22) Filed: Jun. 21, 2004

(51) Int. Cl. A61B 5/05

(52) U.S. Cl. 600/345

(57) ABSTRACT

The present invention provides an instantaneous noninvasive hematocrit determination device adapted for assessing the hematocrit value of a patient. The device comprises at least one pair of electrodes, wherein one one pair of electrodes is adapted to be engaged with a portion of the patient and the other one pair is adapted to be engaged with another portion of the patient's body and wherein each the pair of electrodes comprises a current electrode and a potential electrode. The device further comprises a harmonic oscillator adapted to vibrate at relatively high frequencies and at relatively low frequencies, a stable current source adapted to receive vibrations from the harmonic oscillator and produce a relatively weak alternating amplitude stable current that is adapted to pass through the current electrode. The device further comprises at least one detector adapted to detect the signal received by the potential electrode and a calculating machine adapted to process the hematocrit according to the equation 0.6·H²+(A−2.4)·H+1.6−1.6·A=0 wherein H is the hematocrit and A is an alternating components of the resistance value of the patients body at the relatively high frequencies and at the relatively low frequencies as well as an indicator adapted to indicate the hematocrit of the patient. whereby stable current is transmitted to the patient's body through the current electrodes and the voltage drop in the body is measured by the potential electrodes and the hematocrit is calculated by said calculating machine so as to indicate the hematocrit value in any instant.

### FIELD OF THE INVENTION

The present invention relates to hematology. More particularly, the present invention relates to an automated and controlled real time determination of hematocrit values.

### BACKGROUND OF THE INVENTION

Erythrocytes amount assessment is a known method for evaluating blood parameters and is commonly called hematocrit. The method consists of taking a blood sample, mixing it with heparin and isotonic solution of NaCl, and separating the erythrocytes from the blood plasma using a centrifuge. The erythrocytes are mixed with isotonic solution after draining the plasma. Ultrasound velocities passing through the sample is measured and the difference between the ultrasound velocity in the sample and the isotonic solution is determined using an ultrasound interferometer. Then, the amount of erythrocytes X is obtained from the equation:
X=K·(Cₐ−Q₆)·A
wherein K is an empiric constant; A is the degree of sample dilution; (Cₐ−C₆) is the velocity of ultrasound is the difference between the ultrasound velocity in the sample and the isotonic solution, respectively. Determining the hematocrit using this method requires a centrifuge and a differential digital interferometer. This method disadvantage is that it is an invasive method that requires using expensive equipment, the centrifuge and the interferometer. Another drawback of the method is that the user has to send the blood sample to a laboratory and then wait for a result that takes valuable time.

It any of the ways that are provided in order to measure or assess the hematocrit in a patient's blood, the measurement is an invasive measurement in which blood has to be withdrawn and it is a measurement which results are not immediate and there is a predetermined time in which the separation of erythrocytes from the plasma takes place. There is no method or device in which hematocrit can be measures in an on-line manner in which results of the hematocrit value are instantaneously obtained.

A method has been developed based on the electrical conductivity of the blood. This method is disclosed in Romanov U. V., Leus V. I and Andreev V. C., Method for calculating number of hematocrit, Laboratory 1973, vol. 8, pp. 451 (document in Russian). According to the method described, the conductometric device comprises a capillary tube and a metal container. A blood sample is received in the metal container and an electrode system is connected to the device. The electrical conductivities of the blood and plasma are measured and the hematocrit value Hₖ is determined from the following equation obtained from the Maxwell's formula:

$${H}_{k} = \frac{1 - {K}_{κp} / {K}_{nn}}{1 + 0 , 8 \cdot {K}_{κp} / {K}_{nn}} ,$$

wherein Kₖₚ is a value proportional to the blood electrical conductivity; Kₙₙ is a value proportional to the plasma electrical conductivity.

This method and device have drawbacks. This device again necessitates the withdrawal of blood from the patient since the electric conductivity of the blood sample is being measured. Another drawback is the lack of possibility to assess the hematocrit in real time.

### SUMMARY OF THE INVENTION

It is an object of the present invention to provide a hematocrit assessment device that is noninvasive.

It is another object of the present invention to provide a hematocrit assessment device that provides real time and instantaneous results without the necessity to wait for results from a laboratory.

Another object of the present invention is to decrease the traumatic effect of blood withdrawal, and simplification of the method of hematocrit assessment.

It is yet another object of the present invention to provide a method of assessing hematocrit in the blood. The method described is a noninvasive method that provides instantaneous results in real time.

In addition, it is an object of the present invention to provide a device for assessing the hematocrit in the blood incorporated with an assessment of fluid accumulated in different areas of the body.

It is therefore provided in accordance with a preferred embodiment of the present invention an instantaneous noninvasive hematocrit determination device adapted for assessing the hematocrit value of a patient, said device comprising:
at least one pair of electrodes, wherein one of said at least one pair of electrodes is adapted to be engaged with a portion of the patient and the other one of said at least one pair of electrodes is adapted to be engaged with another portion of the patient's body and wherein each one of said at least one pair of electrodes comprises a current electrode and a potential electrode;
a harmonic oscillator adapted to vibrate at relatively high frequencies and at relatively low frequencies;
a stable current source adapted to receive vibrations from said harmonic oscillator and produce a relatively weak alternating amplitude stable current that is adapted to pass through said current electrode;
at least one detector adapted to detect the signal received by said potential electrode;
calculating machine adapted to process the hematocrit according to equation 0.6·H²+(A−2.4)·H+1.6−1.6·A=0 wherein H is the hematocrit and A is an alternating components of the resistance value of the patient's body at said relatively high frequencies and at said relatively low frequencies;
indicator adapted to indicate the hematocrit of the patient.
   whereby stable current is transmitted to the patient's body through the current electrodes and the voltage drop in the body is measured by the potential electrodes and the hematocrit is calculated by said calculating machine so as to indicate the hematocrit value in any instant.

Furthermore, in accordance with another preferred embodiment of the present invention, said resistance value is evaluated by a model of the blood vessel resistance.

Furthermore, in accordance with another preferred embodiment of the present invention, at least one filter connected to said is adapted to filter the signal said at least one detector.

Furthermore, in accordance with another preferred embodiment of the present invention, said low frequencies are 20-40 kHz.

Furthermore, in accordance with another preferred embodiment of the present invention, said high frequencies are 200-400 kHz.

Furthermore, in accordance with another preferred embodiment of the present invention, said indicator is a LED display.

Furthermore, in accordance with another preferred embodiment of the present invention, said calculating machine is a single chip microcontroller.

Furthermore, in accordance with another preferred embodiment of the present invention, said single chip microcontroller is provided with a digital-to-digital converter.

Furthermore, in accordance with another preferred embodiment of the present invention, said oscillator is a digital harmonic oscillator.

Furthermore, in accordance with another preferred embodiment of the present invention, said digital harmonic oscillator produces sinusoidal vibrations.

Furthermore, in accordance with another preferred embodiment of the present invention, amplifiers are provided so as to amplify signal received in the detector.

Furthermore, in accordance with another preferred embodiment of the present invention, said members are wrappers that are adapted to envelope a portion of the body.

Furthermore, in accordance with another preferred embodiment of the present invention, said portion of the body is an arm of the patient.

Furthermore, in accordance with another preferred embodiment of the present invention, said portion of the patient is an ankle.

Furthermore, in accordance with another preferred embodiment of the present invention, said at least one pair of electrodes is made of NiAg.

In addition, it is also provided in accordance with yet another preferred embodiment of the present invention, an instantaneous noninvasive hematocrit determination method adapted for assessing the hematocrit value of a patient, said method comprising:
providing at least one pair of electrodes, wherein each one of said at least one pair of electrodes comprises a current electrode and a potential electrode;
placing one of said at least one pair of electrodes on a body portion of said patient;
placing another one of said at least two [pair of electrodes on another body portion of the patient;
providing a harmonic oscillator that is adapted to vibrate at relatively high frequencies and at relatively low frequencies;
providing a stable current source adapted to receive vibrations from said harmonic oscillator and produce a relatively weak alternating amplitude stable current that is adapted to pass through said current electrode;
providing at least one detector adapted to detect the signal received by said potential electrode;
providing a calculating machine adapted to process the hematocrit according to equation 0.6·H²+(A−2.4)·H+1.6−1.6·A=0 wherein H is the hematocrit and A is an alternating components of the resistance value of the patient's body at said relatively high frequencies and at said relatively low frequencies;
providing an indicator adapted to indicate the calculated hematocrit.
   whereby stable current is transmitted to the patient's body through the current electrodes and the voltage drop in the body is measured by the potential electrodes and the hematocrit is calculated by said calculating machine so as to indicate the hematocrit value in any instant.

### BRIEF DESCRIPTION OF THE FIGURES

In order to better understand the present invention and appreciate its practical applications, the following Figures are attached and referenced herein. Like components are denoted by like reference numerals.

It should be noted that the figures are given as examples and preferred embodiments only and in no way limit the scope of the present invention as defined in the appending Description and Claims.

FIG. 1 illustrates a schematic representation of the equivalent electrical circuit analogous to a blood vessel part in accordance with a preferred embodiment of the present invention.

FIG. 2 illustrates a schematic representation of a hematocrit assessment device in accordance with a preferred embodiment of the present invention.

FIG. 3 illustrates schematic illustration of a hematocrit assessment device in accordance with a preferred embodiment of the present invention.

FIG. 4 illustrates a comparison between hematocrit measurements using a standard invasive method and hematocrit assessment using a hematocrit assessment device in accordance with a preferred embodiment of the present invention in three patients.

### DETAILED DESCRIPTION OF THE INVENTION AND THE FIGURES

The present invention provides a novel and unique method and device for hematoctrit assessment. The hematocrit assessment device is a noninvasive device that is based on conductometric measurements that provides an assessment in an instantaneous manner.

The aim of the disclosed present invention is decreasing of the traumatic effect involved in hematocrit assessments as made today in which blood is withdrawn, simplification of the method and assessment of the hematocrit value in real time.

Basically, the problem of hematocrit assessment is solved by measuring the blood electrical conductivity in a noninvasive manner. In one aspect of the present invention, electrodes are attached to a wrist and an ankle of a patient, however, the electrodes can be attached on two wrists, two ankles or any other body parts so as to allow an electric circuit to pass through a body portion. The alternating component of the body resistance value correlated with the blood electrical conductivity is measured at frequencies of about 20-40 kHz and 200-400 kHz. Then, the value of hematocrit H is determined from the following equation
0.6·H²+(A−2.4)·H+1.6−1.6·A=0, (1)
wherein A is the ratio between the alternating components of the body resistance value at frequencies of 200-400 kHz and 20-40 kHz, accordingly. Since mathematically, there are two solutions to equation (1), the solution that is chosen is that one that corresponds the physiological range of hematocrit in the blood. The other solution that exceeds the limits of the physiologic range is discarded.

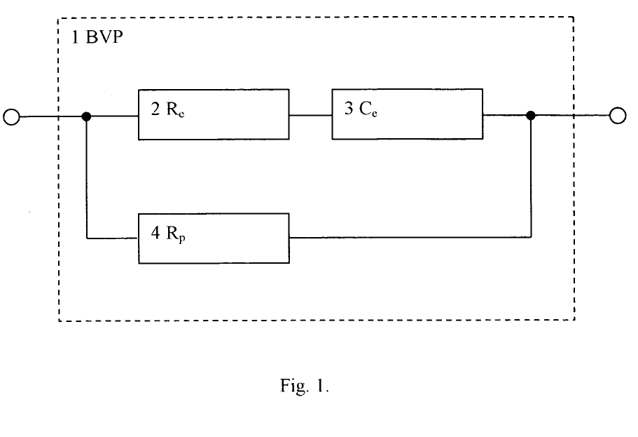

Reference is now made to FIG. 1 illustrating a schematic representation of the equivalent electrical circuit analogous to a blood vessel part in accordance with a preferred embodiment of the present invention. This electric circuit represents a model of blood vessel resistance. It is a well known fact that the alternating component of the human body's resistance is governed mainly by the blood electrical conductivity and by blood pulse volume. Pulse volume is not dependent on the frequency of a current, however, the ratio between alternating components of the body resistance value at frequencies of 200-400 kHz and 20-40 kHz is governed solely by the electrical properties of the blood. At frequency of 20-40 kHz, an alternating current flows mainly through vessels because of cell membrane capacitance, so that the blood electrical conductivity is the plasma electrical conductivity. At frequency of 200-400 kHz, both erythrocytes and plasma conduct current. The equation for assessing hematocrit shown herein before (1) is obtained from a mathematical model, describing blood as heterogeneous system.

The equation based on Bruggemann's theory describing a dependence of the hematocrit value on the blood electrical conductivity at frequencies of 20-40 kHz was obtained by De La Rue and Tobias and is as follows:
σ₁=σₚ·(1−H)^{3/2}, (2)
wherein σ₁ is the blood electrical conductivity at low frequencies, σₚ is the plasma electrical conductivity, and H is the hematocrit value.

At 200-400 kHz, the dependence of the hematocrit value from the blood electrical conductivity is described by the following equation:
σ₂=σₚ·(1−H)+σₑ·H, (3)
wherein σ₂ is the blood electrical conductivity at high frequencies, σₚ is the plasma electrical conductivity, σₑ is the electrical conductivity of cytoplasm of erythrocytes, and H is the hematocrit value. Equation (3) was obtained using the equivalent electrical circuit analogous to a blood vessel part shown in FIG. 1. The model of a blood vessel part BVP (designated by 1) consists of erythrocytes' cytoplasm Rₑ (2), erythrocytes' membranes Cₑ (3), and blood plasma Rₚ (4). When an alternating current at 200-400 kHz passes through the blood, the electrical conductivity of BVP (1) is equal to the sum of electrical conductivities of erythrocytes' cytoplasm Rₑ (2) and blood plasma Rₚ (4) in proportions on their volumes, as the effect of erythrocytes' membranes Cₑ (3) diminishes to insignificant proportions.

Typical values of σₑ and σₚ are known. It is also known that the electrical conductivity of the plasma is a frequency independent value. The inventors of the present invention expanded in Taylor row the right part of equation (2) and limited it to the first three terms, than divided equation (2) by equation (3), and obtained the equation (1) through simple mathematical transformations.

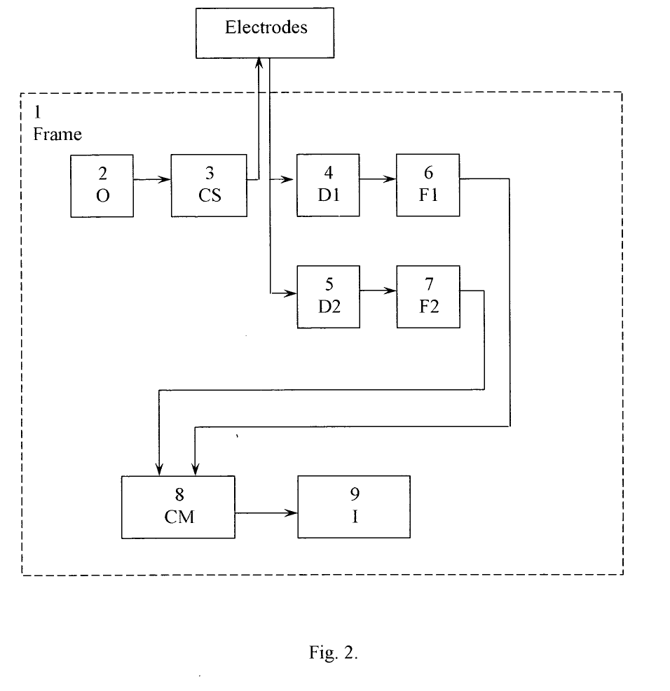

Reference is now made to FIG. 2 illustrating a schematic representation of a hematocrit assessment device in accordance with a preferred embodiment of the present invention. Hematocrit assessment device comprises of a frame 1 that is provided with harmonic oscillator 2 (O), stable current source 3 (CS), two detectors 4 (D1) and 5 (D2), two filters 6 (F1) and 7 (F2), calculating machine 8 (CM) and indicator 9 (I).

Harmonic oscillator 2 (O) is connected in series to stable current source 3 (CS), wherein both work at frequencies of 20-40 kHz and 200-400 kHz at the same time. It should be mentioned that in case a conventional oscillator that can produce oscillations solely in one frequency at a time, two oscillators are to be used. Harmonic oscillator 2 is preferably a generator that uses direct digital synthesis. This method allows to form a signal of complex spectral structure that includes the sum of two sinusoids in two different frequencies. Basically, using this method, the generator generates a sequence of pulses having amplitudes of +V, 0, −V and calculation of duration arrangements. As a result, the spectral structure of a target pulse sequence is formed. In this case, the result is the sum of harmonics of two frequencies with a minimum level of parasitic harmonics. Detector 4 (D1) is connected in series with filter 6 (F1), wherein detector 4 and filter 6 work at frequency of 20-40 kHz. Detector (D2) is connected in series to filter 7 (F2), wherein both detector 5 and filter 7 work at frequency of 200-400 kHz. The output of stable current source 3 (CS) is received as an input to detectors 4 (D1) and 5 (D2) through electrodes 10. The outputs of filters 6 (F1) and 7 (F2) are electrically connected to inputs of calculating machine 8 (CM), and the output of calculating machine 8 (CM) is connected to indicator 9 (I).

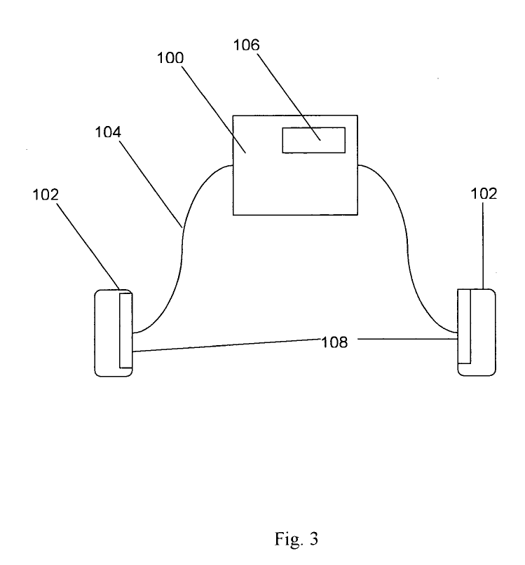

Optionally, a LED display can be used as indicator 9 (I). Optionally, a single chip microcontroller with analog-to-digital converter may be used as calculating machine 8 (CM). Optionally, a digital oscillator using a direct synthesis method may be used as harmonic oscillator 2 (O). Operational amplifiers can be used in order to realize other functional blocks in FIG. 2. The examples of functional operators given herein are set for an example only and in no way limit the scope of the present invention. An amplitude-modulated signal obtained from sinusoidal vibrations at frequency of about 20-40 kHz and about 200-400 kHz from harmonic oscillator 2 (O), is transmitted to stable current source 3 (CS), from which a weak alternating amplitude-stabled current passes through electrodes 10. In a preferred embodiment of the present invention, tetrapolar reography is used; therefore electrodes 10 represent two pair of electrodes that are being engaged with the patient's body. The first pair are outer electrodes that are current electrodes and are the electrodes that pass the stable current from current source 3 to the patient's body. The voltage drop across the body is measured using the other pair of electrodes that are inner electrodes (potential electrodes) from which the signal passes to the inputs of the detectors 4 (D1) and 5 (D2) (both pairs of electrodes are shown in FIG. 3).

The inputs are amplified and detected in detectors 4 and 5. The detected signals output from detectors 4 (D1) and 5 (D2) and pass as inputs to filters 6 (F1) and 7 (F2). The inputs that are proportional to the alternating component of the body resistance value signals at 20-40 kHz and 200-400 kHz are filtered and extra amplified. The signals that output of filters 6 (F1) and 7 (F2) is transmitted to calculating machine 8 (CM), where analog signals are converted to digital signals, and the ratio between alternating components of the body resistance value at 200-400 kHz and 20-40 kHz is determined. The hematocrit value H is obtained from by equation (1) and displayed in indicator 9 (I).

Reference is now made to FIG. 3 illustrating a schematic illustration of a hematocrit assessment device in accordance with a preferred embodiment of the present invention. Hematocrit automated determination device 100 in which the components shown in frame 1 of FIG. 2 are accommodated is electrically connected by two pairs of wires 104 to two pairs of electrodes 108 and 110, wherein each pair is embedded in one of two members 102. As mentioned herein before, tetrapolar reography is used so that one pair of electrode transmit a current to the body of the patient and the other pair measures the drop of voltage through the body. Members 102 can be a wrapper that envelopes an arm so as to achieve good conductivity between the skin and the electrodes. In order to achieve conductivity, the member has to be wet when in use. Each member can be placed on an arm or on a leg. The results of the hematocrit assessment are shown on a display 106.

In an experiment in which the method of the present invention had been examined, NiAg electrodes F9010 were attached to the wrist and to the ankle of a patient. The hematocrit value was measured using the hematocrit assessment device shown in FIG. 3 and the following results were obtained at frequency limits as indicated herein:

1. At f₁=20 kHz and f₂=200 kHz H=0,497;

2. At f₁=40 kHz and f₂=400 kHz H=0,493.

It has been shown that the real time hematocrit assessment device provides accurate results for the hematocrit value in the blood.

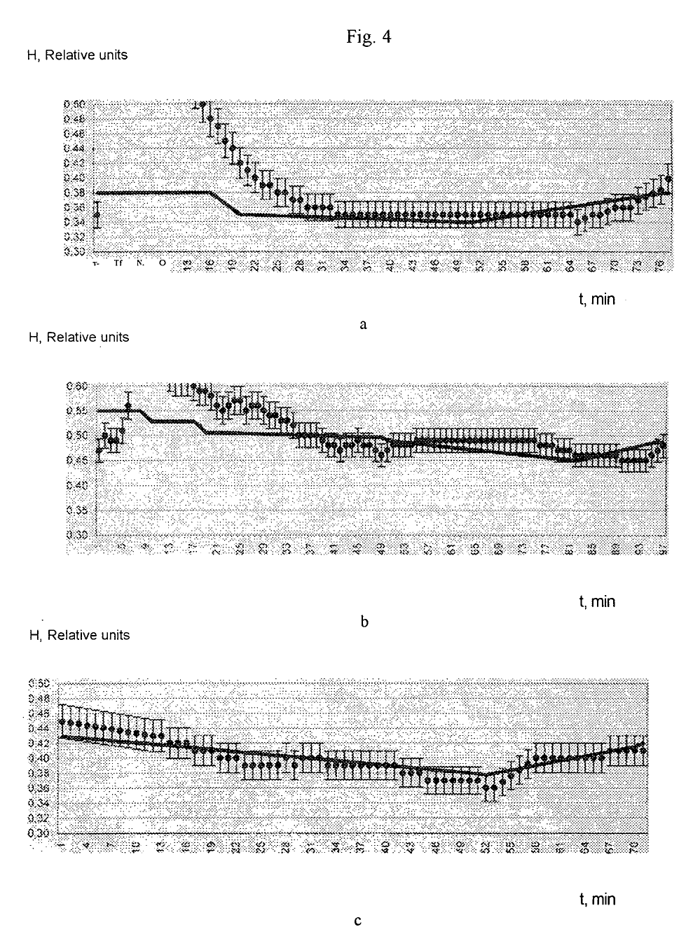

Reference is now made to FIG. 4 illustrating a comparison between hematocrit measurements using a standard invasive method and hematocrit assessment using a hematocrit assessment device in accordance with a preferred embodiment of the present invention in three patients. Each patient is represented in another graph (a, b, and c). The real measurements of hematocrit assessments were taken after 25 min and on. The straight line represents measurements of hematocrit using a standard method of centrifuging the erythrocytes from the plasma and the dots represents measurements taken by the hematocrit assessment device of the present invention. The standard errors between the two graphs are about 5%, which shows a good correlation between the invasive method and the noninvasive method disclosed herein.

It should be mentioned that the method disclosed herein is a noninvasive method that uses solely conductance measurements and the results are instantaneous and continuous results without the need to send blood to a laboratory.

It should be clear that the description of the embodiments and attached Figures set forth in this specification serves only for a better understanding of the invention, without limiting its scope as covered by the following Claims.

It should also be clear that a person skilled in the art, after reading the present specification can make adjustments or amendments to the attached Figures and above described embodiments that would still be covered by the following Claims.

1. An instantaneous noninvasive hematocrit determination device adapted for assessing the hematocrit value of a patient, said device comprising:
at least one pair of electrodes, wherein one of said at least one pair of electrodes is adapted to be engaged with a portion of the patient and the other one of said at least one pair of electrodes is adapted to be engaged with another portion of the patient's body and wherein each one of said at least one pair of electrodes comprises a current electrode and a potential electrode;
a harmonic oscillator adapted to vibrate at relatively high frequencies and at relatively low frequencies;
a stable current source adapted to receive vibrations from said harmonic oscillator and produce a relatively weak alternating amplitude stable current that is adapted to pass through said current electrode;
at least one detector adapted to detect the signal received by said potential electrode;
calculating machine adapted to process the hematocrit according to equation 0.6·H²+(A−2.4)·H+1.6−1.6·A=0 wherein H is the hematocrit and A is an alternating components of the resistance value of the patient's body at said relatively high frequencies and at said relatively low frequencies;
indicator adapted to indicate the hematocrit of the patient.
whereby stable current is transmitted to the patient's body through the current electrodes and the voltage drop in the body is measured by the potential electrodes and the hematocrit is calculated by said calculating machine so as to indicate the hematocrit value in any instant.

2. The device as claimed in claim 1, wherein said resistance value is evaluated by a model of the blood vessel resistance.

3. The device as claimed in claim 1, wherein at least one filter connected to said is adapted to filter the signal said at least one detector.

4. The device as claimed in claim 1, wherein said low frequencies are 20-40 kHz.

5. The device as claimed in claim 1, wherein said high frequencies are 200-400 kHz.

6. The device as claimed in claim 1, wherein said indicator is a LED display.

7. The device as claimed in claim 1, wherein said calculating machine is a single chip microcontroller.

8. The device as claimed in claim 7, wherein said single chip microcontroller is provided with a digital-to-digital converter.

9. The device as claimed in claim 1, wherein said oscillator is a digital harmonic oscillator.

10. The device as claimed in claim 9, wherein said digital harmonic oscillator produces sinusoidal vibrations.

11. The device as claimed in claim 1, wherein amplifiers are provided so as to amplify signal received in the detector.

12. The device as claimed in claim 1, wherein said members are wrappers that are adapted to envelope a portion of the body.

13. The device as claimed in claim 1, wherein said portion of the body is an arm of the patient.

14. The device as claimed in claim 1, wherein said portion of the patient is an ankle.

15. The device as claimed in claim 1, wherein said at least one pair of electrodes is made of NiAg.

16. An instantaneous noninvasive hematocrit determination method adapted for assessing the hematocrit value of a patient, said method comprising:
providing at least one pair of electrodes, wherein each one of said at least one pair of electrodes comprises a current electrode and a potential electrode;
placing one of said at least one pair of electrodes on a body portion of said patient;
placing another one of said at least two [pair of electrodes on another body portion of the patient;
providing a harmonic oscillator that is adapted to vibrate at relatively high frequencies and at relatively low frequencies;
providing a stable current source adapted to receive vibrations from said harmonic oscillator and produce a relatively weak alternating amplitude stable current that is adapted to pass through said current electrode;
providing at least one detector adapted to detect the signal received by said potential electrode;
providing a calculating machine adapted to process the hematocrit according to equation 0.6·H²+(A−2.4)·H+1.6−1.6·A=0 wherein H is the hematocrit and A is an alternating components of the resistance value of the patient's body at said relatively high frequencies and at said relatively low frequencies;
providing an indicator adapted to indicate the calculated hematocrit. whereby stable current is transmitted to the patient's body through the current electrodes and the voltage drop in the body is measured by the potential electrodes and the hematocrit is calculated by said calculating machine so as to indicate the hematocrit value in any instant.
